(19) United States
(12) Patent Application Publication
(10) Pub. No.: US 2005/0079194 A1
(43) Pub. Date: Apr. 14, 2005

(54) Cosmetic compositions comprising alk(en)ylsuccinic acid derivatives

(75) Inventors: Peter Klug, Grossostheim (DE); Torsten Henning, Bad Soden (DE); Franz Xaver Scherl, Burgkirchen (DE)

Correspondence Address: CLARIANT CORPORATION;Industrial Property Department, 4000 Monroe Road, Charlotte, NC, 28205, US

(73) Assignee: Clariant GmbH

(21) Appl. No.: 10/999,479

(22) Filed: Nov. 30, 2004

Related U.S. Application Data
(63) Continuation-in-part of application No. 10/003,656, filed on Nov. 24, 2001.

(30) Foreign Application Priority Data
Nov. 29, 2000 (DE) 100 59 318.6

(51) Int. Cl. A61K 7/46; A61K 7/00

(52) U.S. Cl. 424/401; 512/1

(57) ABSTRACT

The invention relates to cosmetic and pharmaceutical compositions containing a fragrance and at least one emulsifier comprising
a) at least one alkyl chain and/or alkenyl chain having at least 28 carbon atoms obtainable by polymerization of (C₂-C₅)-alkenes and which is linked with b) at least one carboxylic acid, carboxylic acid derivative, carboxylic anhydride, carboxylic anhydride derivative, ester and/or amide group.
   Particularly preferred emulsifiers are alkenylsuccinic anhydrides according to formula (1) and derivatives thereof, 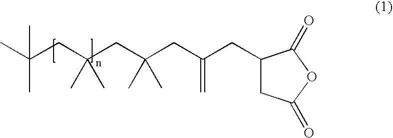 in which n is equal to or greater than 4.

### CROSS-REFERENCE TO RELATED APPLICATIONS

This application is a continuation-in-part application of copending application Ser. No. 10/003,656, filed Nov. 24, 2001, which is hereby incorporated by reference.

### BACKGROUND OF THE INVENTION

To prepare cosmetic compositions, combinations of fatty acid salts and nonionic surfactants are widely used as emulsifiers. A disadvantage associated therewith is the increase in the pH caused by the fatty acid salts, as a result of which the cosmetic compositions are alkaline. Since the surface of the human skin is covered with a slightly acidic membrane (pH 4.5 to 6.5), it is desirable to adjust cosmetics to a slightly acidic or neutral pH. However, the addition of neutralizing agents, such as, for example, hydrochloric acid, citric acid or lactic acid, impairs the emulsifying action of the systems.

In EP 0 553 241, mixtures of alkyl polysaccharides, fatty alcohols and optionally polysaccharides are used to prepare emulsions.

WO 92/07543 describes alkyl glycosides and fatty acid partial glycerides as cosmetic emulsifiers.

Since the nonionic surfactants have only an inadequate emulsifier action, they have to be used in large amounts, thus impairing the skin friendliness of the cosmetic compositions.

As is known, good emulsifying action is shown by ethoxylated fatty acid esters, for example polyethylene glycol stearate (30 EO units) and sorbitan oleate. However, products which contain ethylene oxide are suspected of making the skin pervious to harmful substances and of forming undefined and possibly harmful substances under the action of UV, for which reason they are undesirable for cosmetic products.

In addition to emulsifying components, W/O emulsions usually comprise metal soaps and lipophilic waxes to stabilize the viscosity, for example beeswax, microcrystalline waxes or esters of fatty acids and fatty alcohols with linear or branched fatty residues, for example stearates, wool grease or wool grease derivatives. The disadvantage of this is that, due to the lipophilic waxes which are necessarily present, such emulsions are only available in solid form, i.e. with a melting point or dropping point of from 50 to 60° C.—or where the lipophilic waxes are omitted, have inadequate phase stability and unsatisfactory oil-binding ability.

WO 87/03 613, EP 155 800 and U.S. Pat. No. 4,911,770 describe carboxylic acids, carboxylic anhydrides, ester derivatives and amide derivatives with long-chain (C₂₀-C₅₀₀)-alkyl radicals as emulsifiers for explosives.

### SUMMARY OF THE INVENTION

Surprisingly, it has now been found that emulsifiers comprising
a) at least one alkyl chain and/or alkenyl chain having at least 28 carbon atoms obtainable by polymerization of (C₂-C₅)-alkenes and which is linked with
b) at least one carboxylic acid, carboxylic acid derivative, carboxylic anhydride, carboxylic anhydride derivative, ester and/or amide group, are particularly suitable as emulsifiers for cosmetic and pharmaceutical compositions.

Accordingly, the invention provides cosmetic and pharmaceutical compositions containing at least one emulsifier comprising
a) at least one alkyl chain and/or alkenyl chain having
b) at least 28 carbon atoms obtainable by polymerization of (C₂-C₅)-alkenes and which is linked with at least one carboxylic acid, carboxylic acid derivative, carboxylic anhydride, carboxylic anhydride derivative, ester and/or amide group.

### DETAILED DESCRIPTION OF THE INVENTION

Preferred (C₂-C₅)-alkenes are ethylene, propylene, butene and isobutene, particularly preferably isobutene. The polyalkyl chains and polyalkylene chains may also be copolymers of different (C₂-C₅)-alkenes.

Particularly preferred alkyl chains are polyisobutenyl chains obtainable by polymerization of isobutene.

The alkyl chains and alkenyl chains a) preferably have 28 to 200, particularly preferably 40 to 150, particularly preferably 52 to 100, carbon atoms.

Group b) is preferably a carboxylic acid, carboxylic acid derivative, carboxylic anhydride or carboxylic anhydride derivative group, particularly preferably a carboxylic anhydride or carboxylic anhydride derivative group.

A particularly preferred group b) is a carboxylic anhydride derivative group.

The carboxylic acid, carboxylic acid derivative, carboxylic anhydride and carboxylic anhydride derivative groups are preferably those derived from unsaturated carboxylic acids and carboxylic anhydrides, preferably from acrylic acid, methacrylic acid, maleic acid and anhydrides thereof, particularly preferably maleic acid and/or maleic anhydride.

Particularly preferred emulsifiers are alkenylsuccinic anhydrides according to formula (1) and derivatives thereof, 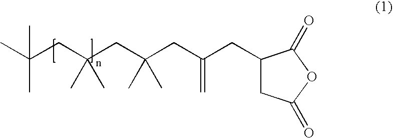
in which n is equal to or greater than 4, preferably 4 to 46, particularly preferably 7 to 35, especially preferably 10 to 22. The alkenylsuccinic anhydrides according to formula (1) can also be present in hydrogenated form, which has an advantageous effect on the oxidation stability. More particularly, derivatives of alkenylsuccinic anhydrides of formula (1) are characterized in the ester form according to formula (2) 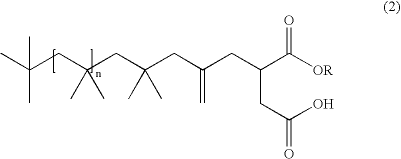
wherein n is equal to or greater than 4, and R is derived from a mono-functional alcohol selected from the group consisting of an ethoxylated monoalcohol, a propoxylated monoalcohol, a fatty alcohol ethoxylate, methyl glycol and methyltriglycol.

The emulsifiers are preferably prepared by ene reaction of the alkenes corresponding to the chains a) and obtainable by polymerization of (C₂ -C₅)-alkenes with the unsaturated carboxylic acids and/or carboxylic anhydrides corresponding to the groups b). Optionally, the chains a) can be hydrogenated. The alkenylsuccinic anhydrides according to formula (1) are preferably obtainable by ene reaction of the corresponding polyisobutenes obtainable by polymerization of isobutene with maleic anhydride.

The synthesis of the emulsifiers can be carried out in accordance with the techniques familiar to the person skilled in the art (see also WO 87/03613, EP 155 800 and U.S. Pat. No. 4,911,770).

Derivation of the carboxylic acid and/or carboxylic anhydride groups b) advantageously takes place after the ene reaction.

The carboxylic acid derivative and carboxylic anhydride derivative groups are preferably those obtainable by reacting the corresponding carboxylic acid and carboxylic anhydride groups with alkali, mono- and polyfunctional alcohols, aminoalcohols, unsubstituted mono- and polyamines and/or substituted mono- and polyamines. Carboxylic anhydride groups, and particularly preferably maleic anhydride groups, are preferably derived. The derivation can also be a mixed derivation.

The carboxylic acid derivative and carboxylic anhydride derivative groups are preferably carboxylic acid salts, mono- and dicarboxylic ester, carboxamide, amino ester and/or imide groups, particularly preferably the corresponding maleic acid derivative groups.

For the derivation, particular preference is given to alk(en)ylsuccinic anhydrides according to formula (1).

For the derivation of alcohols, suitable monofunctional alcohols having 1 to 30 carbon atoms are preferred, preferably methanol, ethanol, propanol, isopropanol, butanol, pentanol, dodecanol and/or octadecanol; ethoxylated and/or propoxylated monoalcohols, preferably methyl glycols, in particular methyl triglycol, ethyl glycol and/or butyl glycol; fatty alcohol ethoxylate; glycerol; polyglycerol; sugar alcohols, preferably sorbitol and sorbitan; ethylene glycol; propylene glycol; oligomers of ethylene glycol; oligomers of propylene glycol; polyalkylene glycols, preferably polyethylene glycols; and/or random and blocklike copolymers of ethylene oxide and propylene oxide.

Particularly preferred alcohols are methyl triglycol, glycerol, polyglycerols, polyethylene glycols and/or copolymers of ethylene oxide and propylene oxide.

In the derivation by reaction with aminoalcohols, either amides, imides or amino esters are formed depending on the amino alcohol. Suitable aminoalcohols are, in principle, all aminoalcohols having a free OH— and/or amine function NH—, particularly suitable are those with a tertiary amino function.

Preferably suitable aminoalcohols are ethanolamine, isopropanolamine, diethanolamine, triethanolamine, diethylethanolamine, dimethylethanolamine and/or dibutylethanolamine, particularly preferably triethanolamine, diethylethanolamine and/or dimethylethanolamine.

In the derivation by reaction with amines, the corresponding amides or imines are formed. Suitable amines are all mono- and polyamines having at least one reactive amino function.

Preferred amines are ammonia, monoalkylamines having a (C₁-C₃₀)-alk(en)yl radical, particularly preferably (C₁-C₁₀)-alk(en)yl radical, diamines having (C₁-C₃₀)-alk(en)yl radicals, particularly preferably (C₁-C₁₀)-alk(en)yl radicals, in particular 3-dimethylaminopropylamine, and/or polyamines, preferably polyethyleneimines.

In the derivation by reaction with alcohols, aminoalcohols and amines, the molar ratio of carboxylic anhydride groups to alcohol, aminoalcohol or amine is in each case preferably 1:0.9 to 1:2, particularly preferably 1:1.

With the particularly preferred ratio of 1:1, the corresponding monoesters or mono-amides form. The free carboxyl groups can also be present in the form of their metal or amine salts.

Particularly preferred emulsifiers are the derivatives obtainable by reacting the alk(en)ylsuccinic anhydrides according to formula (1) with glycerol, triethanolamine, diethylethanolamine, methyl glycols, preferably methyl triglycol, and/or polyalkylene glycols, preferably polyethylene glycols.

As a result of the derivation, the emulsifying properties can be modified and adapted to the requirements. The carboxyl radicals which are then still free can additionally be converted into the corresponding salts by reaction with amine bases or alkali metal and alkaline earth metal hydroxides. This permits the setting of a corresponding pH in the final formulation and modifies the emulsifying properties. It is thus possible to provide suitable emulsifiers for numerous oils, fats, waxes, paraffins and for water-immiscible solvents and active ingredients in the cosmetics and pharmaceutical sector.

The compositions according to the invention comprise, based on the finished compositions, preferably 0.1 to 8% by weight, particularly preferably 0.3 to 5% by weight, especially preferably 0.5 to 4% by weight, of emulsifiers.

The compositions are preferably oil-in-water emulsions or water-in-oil emulsions.

The nonaqueous fraction of the emulsions, which is largely composed of the emulsifier and the oily substance, is preferably 5 to 95% by weight, particularly preferably 15 to 75% by weight.

The water content of the emulsions varies depending on the desired viscosity of the emulsions. For example, lotions have a comparatively low viscosity, while creams and ointments have a comparatively high viscosity.

Suitable oil substances are preferably Guerbet alcohols having 6 to 18, preferably 8 to 10, carbon atoms; esters of linear and branched (C₆-C₁₃)-fatty acids with linear (C₆-C₂₀)-fatty alcohols; esters of linear (C₆-C₁₈)-fatty acids with branched alcohols, preferably 2-ethylhexanol; esters of linear and branched fatty acids with polyhydric alcohols, preferably dimerdiol and trimerdiol and/or Guerbet alcohols; triglycerides based on (C₆-C₁₀)-fatty acids; vegetable oils, branched primary alcohols; substituted cyclohexanes; Guerbet carbonates; dialkyl ethers; aliphatic hydrocarbons, aromatic hydrocarbons; silicone oils and/or silicone oil derivatives.

As auxiliaries and additives, the compositions can comprise coemulsifiers, superfatting agents, fats, waxes, stabilizers, biogenic active ingredients, light protection substances, UV light protection filters, pigments, metal oxides, micropigments, antioxidants, hydrotropic agents, solubilizers, bodying agents, cationic polymers, glycerol, preservatives, dispersants, protein derivatives, such as, for example, gelatin and collagen hydrolysates, natural- and synthetic-based polypeptides, egg yolk, lecithin, lanolin, lanolin derivatives, fatty alcohols, silicones, deodorizing agents, substances with a keratolytic and keratoplastic action, enzymes, carrier substances, moisture-donating substances, antimicrobial agents, pearlizing agents, dyes and/or fragrances.

Suitable nonionogenic O/W coemulsifiers are preferably addition products of from 2 to 30 mol of ethylene oxide and/or 0 to 5 mol of propylene oxide with linear fatty alcohols having 8 to 22 carbon atoms, with fatty acids having 12 to 22 carbon atoms and with alkylphenols having 8 to 15 carbon atoms in the alkyl group; (C₁₂-C₁₈)-fatty acid mono-and diesters of addition products of from 1 to 30 mol of ethylene oxide with glycerol; glycerol mono- and diesters and sorbitan mono- and diesters of saturated and unsaturated fatty acids having 6 to 22 carbon atoms and ethylene oxide addition products thereof; addition products of from 15 to 60 mol of ethylene oxide with castor oil and/or hydrogenated castor oil; polyols, in particular polyglycerol esters, such as, for example, polyglycerol polyricinoleate and polyglycerol poly-12-hydroxystearate. Also suitable are mixtures of compounds of two or more of these classes of substance. The addition products of ethylene oxide and/or propylene oxide with fatty alcohols, fatty acids, castor oil and alkylphenols, and the glycerol mono- and diesters of sorbitan mono- and diesters of fatty acids are known, commercially available products. These are mixtures of homologs whose average degree of alkoxylation corresponds to the ratio of the starting amounts of ethylene oxide, propylene oxide and substrate.

(C₁₂-C₁₈)-fatty acid mono- and diesters of addition products of ethylene oxide with glycerol are known from DE-20 24 051 as refatting agents for cosmetic preparations.

The superfattiny agents used are preferably polyethoxylated lanolin derivatives, lecithin derivatives, polyol fatty acid esters, monoglycerides and fatty acid alkanolamides, the latter also serving as foam stabilizers.

Preferred fats are glycerides. Preferred waxes are beeswax, paraffin wax or microcrystalline waxes, optionally in combination with hydrophilic waxes, such as, for example, cetylstearyl alcohol.

Preferred stabilizers are metal salts of fatty acids, such as, for example, magnesium stearate, aluminum stearate and/or zinc stearate.

Biogenic active ingredients are understood as meaning, for example, plant extracts and vitamin complexes.

Suitable preservatives are, for example, phenoxyethanol, formaldehyde solution, parabens, pentanediol or sorbic acid.

Examples of suitable UV filters are 4-aminobenzoic acid,

3-(4′-trimethylammonium)benzylidenebornan-2-one methylsulfate, 3,3,5-trimethylcyclohexyl salicylate; 2-hydroxy-4-methoxybenzophenone; 2-phenylbenzimidazole-5-sulfonic acid and its potassium, sodium and triethanolamine salts; 3,3′-(1,4-phenylenedimethine)bis(7,7-dimethyl-2-oxobicyclo[2.2.1]heptane-1-methanesulfonic acid and its salts; 1-(4-tert-butylphenyl)-3-(4-methoxyphenyl)propane-1,3-dione; 3-(4′-sulfo)benzylideneboman-2-one and its salts; 2-ethylhexyl 2-cyano-3,3-diphenylacrylate; polymers of N-[2(and 4)-(2-oxoborn-3-ylidenemethyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxycarbonyl)anilino]-1,3,5-triazine; 2-(2H-benzotriazol-2-yl)-4-methyl-6-(2-methyl-3-(1 ,3,3,3-tetramethyl-1-(trimethylsilyloxy)disiloxanyl)propyl)phenol; bis(2-ethylhexyl) 4,4′-[(6-[4-((1,1 -dimethylethyl)aminocarbonyl)phenylamino]-1,3,5-triazin-2,4-yl)diimino]bis(benzoate); 3-(4′-methylbenzylidene)-D,L-camphor; 3-benzylidenecamphor; 2-ethylhexyl salicylate; 2-ethylhexyl 4-dimethylaminobenzoate; hydroxy-4-methoxybenzophenone-5-sulfonic acid (sulisobenzonum) and sodium salt thereof, and/or 4-isopropylbenzyl salicylate.

Pigments/micropigments which can be used are, for example, microfine titanium dioxide, zinc oxide and silicon oxide.

Suitable antioxidants are, for example, superoxide dismutase, tocopherol (vitamin E) and ascorbic acid (vitamin C).

The dyes which can be used are the substances approved and suitable for cosmetic and pharmaceutical compositions.

Particularly suitable as thickeners and dispersants are ethylene glycol esters of fatty acids having 14 to 22, particularly preferably 16 to 22, carbon atoms, in particular mono- and diethylene glycol stearate.

Likewise preferably suitable are stearin monoethanolamide, stearin diethanolamide, stearin isopropanolamide, stearin monoethanolamide stearate, stearyl stearate, cetyl palmitate, glyceryl stearate, stearamide diethanolamide distearate, stearamide monoethanolamide stearate, N,N-dihydrocarbyl-(C₁₂-C₂₂)-amidobenzoic acid and soluble salts thereof, N,N-dihydrocarbyl-(C₁₆-C₁₈)-amidobenzoic acid and soluble salts thereof and N,N-di(C₁₆-C₁₈)-amidobenzoic acid and derivatives thereof. Also particularly suitable are polyacrylates and carbomers, in particular those of water-soluble or water-swellable copolymers based on alkylamidoalkylsulfonic acids and N-vinylcarboxamides.

Suitable solubilizers are, in principle, all mono- or polyhydric alcohols and ethoxylated alcohols. Preference is given to using alcohols having 1 to 4 carbon atoms, such as, for example, ethanol, propanol, isopropanol, n-butanol and isobutanol, glycerol and mixtures thereof. Also preferred are polyethylene glycols with a relative molecular mass below 2000. Particular preference is given to polyethylene glycols with a relative molecular mass between 200 and 600 in amounts up to 45% by weight and polyethylene glycols with a relative molecular mass between 400 and 600 in amounts of from 0.5 to 15% by weight.

Further suitable solvents are, for example, triacetin (glycerol triacetate) and 1-methoxy-2-propanol.

Examples of suitable carrier materials are vegetable oils, natural and hydrogenated oils, waxes, fats, water, alcohols, polyols, glycerol, glycerides, liquid paraffins, liquid fatty alcohols, sterols, polyethylene glycols, cellulose and cellulose derivatives.

Suitable fungicidal active ingredients are preferably ketoconazole, oxiconazole, terbinafine, bifonazole, butoconazole, cloconazole, clotrimazole, econazole, enilconazole, fenticonazole, isoconazole, miconazole, sulconazole, tioconazole fluconazole, itraconazole, terconazole, naftifine, Zn pyrethione and octopyrox.

Suitable cationic polymers are, for example, cationic cellulose derivatives, cationic starch; copolymers of diallylammonium salts and acrylamides; quaternized vinylpyrrolidone/vinylimidazole polymers; condensation products of polyglycols and amines; quaternized collagen polypeptides; quatemized wheat polypeptides; polyethyleneimines; cationic silicone polymers, such as, for example, amidomethicones; copolymers of adipic acid and dimethylaminohydroxy-propyldiethylenetriamine; polyaminopolyamide and cationic chitin derivatives, such as, for example, chitosan. Suitable silicone compounds are, for example, dimethylpolysiloxane, methylphenylpolysiloxanes, cyclic silicones and amino-, fatty acid-, alcohol-, polyether-, epoxy-, fluorine-and/or alkyl-modified silicone compounds, and polyalkylsiloxanes, polyalkylarylsiloxanes and polyether siloxane copolymers—as described in U.S. Pat. No. 5,104,645 and the publications cited therein—which at room temperature may be either in liquid form or else in resin form.

The compositions according to the invention can be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances.

Examples of available moisture-donating substances are isopropyl palmitate, glycerol and/or sorbitol, which are preferably used in amounts of from 0.1 to 50% by weight.

Deodorizing substances which can be used are, for example, allantoin and bisabolol, preferably in amounts of from 0.0001 to 10% by weight.

Suitable pearlizing agents are, for example, glycol distearic esters, such as, for example, ethylene glycol distearate, and also fatty acid monoglycol esters.

The total proportion of auxiliaries and additives is, based on the finished compositions, preferably 1 to 10% by weight, particularly preferably 2 to 5% by weight.

In the case of emulsions according to the invention, the preparation can be carried out in a manner known per se, i.e. for example by hot, cold, hot-hot/cold or PIT emulsification.

The compositions according to the invention are characterized by good high-temperature and low-temperature stability, high water-absorbing capacity coupled with a high oil-binding ability, good skin compatibility and good compatibility with the ingredients customary in cosmetic and pharmaceutical compositions. It is particularly advantageous that the compositions according to the invention can be free from lipophilic waxes, such as, for example, beeswax, microcrystalline waxes, hydrogenated castor oil or esters or fatty acids and fatty alcohols with linear or branched fatty radicals, such as, for example, stearates, wool greases and wool grease derivatives, and a high viscosity stability is achieved nevertheless. A further advantage is the high stability of the compositions according to the invention, which can otherwise only be achieved using rather expensive silicone emulsifiers.

The cosmetic and pharmaceutical compositions may, for example, be skincare compositions, such as, for example, daycreams, night creams, care creams, nourishing creams and body lotions; ointments, creams and lotions comprising pharmaceutical active ingredients; deodorant sprays;

deodorant sticks; deodorant gels; decorative cosmetics; sunscreens, aftersun lotions and lotions for the preparation of moistened cleansing and care wipes.

### EXAMPLES

The examples below serve to illustrate the invention in more detail without, however, limiting it thereto.

### Preparation Examples

The base emulsifier used for the examples below was a polyisobutenylsuccinic anhydride with a usable molecular weight of 918.3 g/mol. This was prepared by thermal reaction of ®GIissopal 1000 polyisobutene (BASF) with an excess of 1.5 molar equivalents of maleic anhydride at 205° C., and freed from excess maleic anhydride by vacuum distillation. It is a highly viscous, stringy oil at room temperature.

### Preparation Example 1

Ester of polyisobutenylsuccinic anhydride and glycerol

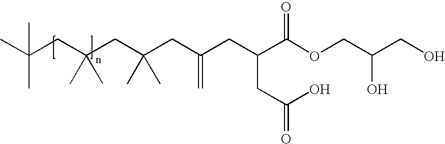

204.6 g of a polyisobutenylsuccinic anhydride with a usable molecular weight of 918.3 g/mol, 87.7 g of paraffin oil (low-viscosity) and 20.5 g of glycerol were introduced under a nitrogen atmosphere, heated to 100° C. and stirred at room temperature for 5 hours. The product was then cooled. This gave a pale yellow oil with an acid number of 37.4 mg of KOH/g.

### Preparation Example 2

Ester of polyisobutenylsuccinic anhydride and triethanolamine

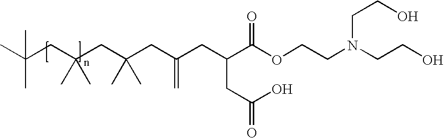

204.4 g of a polyisobutenylsuccinic anhydride with a usable molecular weight of 918.3 glmol, 87.6 g of paraffin oil (low-viscosity) and 33.2 g of triethanolamine were introduced under a nitrogen atmosphere, heated to 100° C. and stirred at this temperature for 5 hours. The product was then cooled. This gave a pale yellow oil with an acid number of 37.2 mg of KOH/g.

### Preparation Example 3

Ester of polyisobutenylsuccinic anhydride and diethylethanolamine

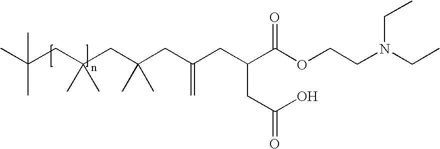

202.0 g of a polyisobutenylsuccinic anhydride with a usable molecular weight of 918.3 g/mol, 86.6 g of paraffin oil (low-viscosity) and 25.8 g of diethylethanolamine were introduced under a nitrogen atmosphere, heated to 100° C. and stirred at this temperature for 5 hours. The product was then cooled. This gave a pale yellow oil with an acid number of 35.4 mg of KOH/g.

### Preparation Example 4

Ester of polyisobutenylsuccinic anhydride and methyl triglycol

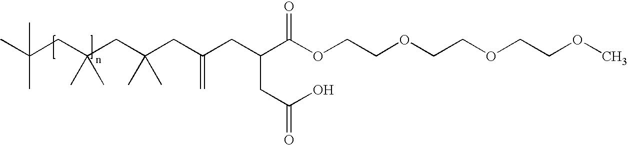

206.1 g of a polyisobutenylsuccinic anhydride with a usable molecular weight of 918.3 g/mol, 88.4 g of paraffin oil (low-viscosity) and 36.1 g of methyl triglycol were introduced under a nitrogen atmosphere, heated to 100° C. and stirred at this temperature for 5 hours. The product was then cooled. This gave a pale yellow oil with an acid number of 37.8 mg of KOH/g.

### Preparation Example 5

Ester of polyisobutenylsuccinic anhydride and polyethylene glycol 200

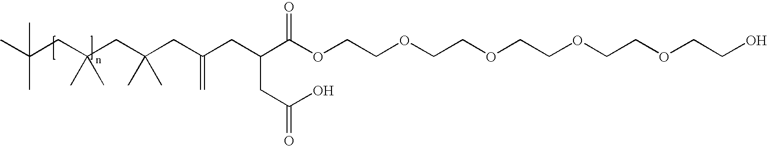

205.0 g of a polyisobutenylsuccinic anhydride with a usable molecular weight of 918.3 g/mol, 44.7 g of paraffin oil (low-viscosity) and 107.0 g of polyethylene glycol 200 were introduced under a nitrogen atmosphere, heated to 100° C. and stirred at this temperature for 5 hours. The product was then cooled. This gave a pale yellow oil with an acid number of 33.3 mg of KOH/g.

### Preparation Example 6

Ester of polyisobutenylsuccininc anhydride and Polyglykol M350 (methyl heptaglycol; a=approx. 7)

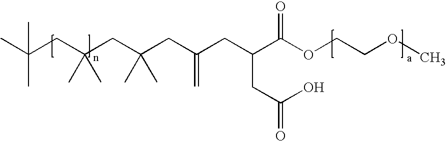

205.0 g of a polyisobutenylsuccinic anhydride with a usable molecular weight of 918.3 g/mol, 121.4 g of Isohexadecane and 78.1 g of Polyglycol M 350 (Methyl heptaglycol with a usable molecular weight of 350 g/mol) were introduced under a nitrogen atmosphere, heated to 100° C. and stirred at this temperature for 5 hours. The product was then cooled. This gave a pale yellow liquid with an acid number of 32.9 mg KOH/g.

### Comparative Example

### Stability of Halfesters of alkenylsuccinic anhydrides

To assess storage stability of the different polyisobutenylsuccinic esters, the acid number of the preparation examples was assessed immediately after preparation and after storage for 1 year. The results indicate, that monofunctional alcohols as shown in the table, Example 4 (methyl triglycol, HO—(CH₂CH₂O)₃—CH₃) and Example 6 (Polyglycol M 350, HO—(CH₂CH₂O)₇—CH₃) exhibit excellent storage stability, whereby the acid numbers essentially did not change in storage, within the error limits. Di- or multifunctional alcohols (polyethylene glycol, glycerol) of Example 5 (di-functional alcohol polyglycol 200, HO—(CH₂CH₂O)₆—OH) and Example 1 (trifunctional alcohol glycerol, HO—CH₂—CHOH—CH₂—OH), respectively, lead to products, which loose approx. 25% of carboxylic acid activity in storage. It is believed that this loss of acid activity in the di- and multi-functional alcohol structure is due to esterification reactions between free OH— and COOH-functionality, which lower the overall free surfactant content, consequently lowering the emulsifier efficacy.

TABLE 1
STABILITY OF POLYISOBUTENYLSUCCINIC ESTERS

| | | | | Acid No |
|---|---|---|---|---|
| | | | Acid No. | after |
| | | | after | storage |
| Example | Alcohol | Functionality | preparation | (1 year) |
| 4 | Methyl triglycol | monofunctional | 37.8 | 37.9 |
| 5 | Polyglykol 200 | difunctional | 33.3 | 25.0 |
| 6 | Polyglykol M | monofunctional | 32.9 | 33.3 |
| | 350 | | | |
| 1 | glycerol | trifunctional | 37.4 | 27.6 |

### Application Examples

### The Percentages are % by Weight

EXAMPLE 1
W/O night cream (without stabilizers such as, for
example, waxes or stearates)

| Phase A | |
|---|---|
| Emulsifier from Preparation example 1 | 1.00% |
| Hostacerin DGI (polyglyceryl-2 sesquiisostearate) | 1.00% |
| Isododecane | 3.00% |
| Isohexadecane | 5.00% |
| Paraffin oil, low-viscosity | 6.25% |
| Isopropyl palmitate | 3.75% |
| Soybean oil | 2.50% |
| Phase B | |
| Water to | 100% |
| Sodium chloride | 0.60% |
| Glycerol | 1.00% |

The oil phase A is heated to 80° C., then the water phase B is added with stirring using a high-speed dispersing tool. The emulsion is stable at a storage temperature of +50° C. for at least 6 weeks. The emulsion is stable after 5 cycles in a swing test (−12° C. /+50° C.).

EXAMPLE 2
W/O cream (without stabilizers such as, for
example, waxes or stearates)

| Phase A | |
|---|---|
| Emulsifiers from Preparation example 2 | 1.00% |
| Isohexadecane | 5.00% |
| Isododecane | 3.00% |
| Paraffin oil, low-viscosity | 6.25% |
| Isopropyl palmitate | 3.75% |
| Soybean oil | 2.50% |
| Phase B | |
| Water to | 100% |
| Sodium chloride | 0.60% |
| Glycerol | 1.00% |

The oil phase A is heated to 80° C., then the water phase B is added with stirring using a high-speed dispersing tool. The emulsion is stable at a storage temperature of +50° C. for at least 6 weeks. The emulsion is stable after 5 cycles in a swing test (−12° C. /+50° C.).

EXAMPLE 3
W/O skin milk (preparable in the cold, without
stabilizers such as, for example, waxes or stearates)

| Phase A | |
|---|---|
| Emulsifier from Preparation example 3 | 1.00% |
| Paraffin oil, low-viscosity | 14.00% |
| Isopropyl palmitate | 5.00% |
| Octyldodecanol | 5.00% |
| Phase B | |
| Water to | 100% |
| Sodium chloride | 2.00% |

The water phase B is added to the oil phase A at room temperature with stirring using a high-speed dispersing tool. The emulsion is stable at a storage temperature of +50° C. for at least 6 weeks. The emulsion is stable after 5 cycles in a swing test (−12° C. /+50° C.).

EXAMPLE 4
W/O soft cream (preparable in the cold, without
stabilizers such as, for example, waxes or stearates)

| Phase A | |
|---|---|
| Emulsifier from Preparation example 4 | 2.00% |
| Hostacerin DGI (polyglyceryl-2 sesquiisostearate) | 1.00% |
| Paraffin oil, low-viscosity | 14.00% |
| Isopropyl palmitate | 5.00% |
| Octyldodecanol | 5.00% |
| Phase B | |
| Water to | 100.00% |
| Sodium chloride | 2.00% |

The water phase B is added to the oil phase A at room temperature with stirring using a high-speed dispersing tool. The emulsion is stable at a storage temperature of +50° C. for at least 6 weeks. The emulsion is stable after 5 cycles in a swing test (−12° C. /+50° C.).

EXAMPLE 5
W/O sunscreen milk (preparable in the cold,
without stabilizers such as, for example, waxes or stearates)

| Phase A | |
|---|---|
| Emulsifier from Preparation example 5 | 2.00% |
| Hostacerin DGI (polyglyceryl-2 sesquiisostearate) | 1.00% |
| Paraffin oil, low-viscosity | 14.00% |
| Isopropyl palmitate | 5.00% |
| Isoamyl p-methoxycinnamate | 4.00% |
| Benzophenone-3 | 1.00% |
| Phase B | |
| Water to | 100.00% |
| Sodium chloride | 2.00% |

The water phase B is added to the oil phase A at room temperature with stirring using a high-speed dispersing tool. The emulsion is stable at a storage temperature of +50° C. for at least 6 weeks. The emulsion is stable after 5 cycles in a swing test (−12° C. /+50° C.).

EXAMPLE 6
W/O sunscreen cream (without stabilizers such as,
for example, waxes or stearates)

| Phase A | |
|---|---|
| Emulsifier from Preparation example 1 | 2.00% |
| Isohexadecane | 5.00% |
| Isododecane | 3.00% |
| Paraffin oil, low-viscosity | 6.25% |
| Isopropyl palmitate | 3.75% |
| Isoamyl p-methoxycinnamate | 4.00% |
| Benzophenone-3 | 1.00% |
| Phase B | |
| Water to | 100% |
| Sodium chloride | 0.60% |
| Glycerol | 1.00% |

The oil phase A is heated to 80° C., then the water phase B is added with stirring using a high-speed dispersing tool. The emulsion is stable at a storage temperature of +50° C. for at least 6 weeks. The emulsion is stable after 5 cycles in a swing test (12° C. /+50° C.).

EXAMPLE 7
W/O baby cream (without stabilizers such as, for
example, waxes or stearates)

| Phase A | |
|---|---|
| Emulsifier from Preparation example 1 | 2.00% |
| Hostacerin DGI (polyglyceryl-2 sesquiisostearate) | 1.00% |
| Isododecane | 3.00% |
| Isohexadecane | 5.00% |
| Paraffin oil, low-viscosity | 6.25% |
| Isopropyl palmitate | 3.75% |
| Phase B | |
| Talc | 10.00% |
| Zinc oxide | 10.00% |
| Phase C | |
| Water to | 100% |
| Sodium chloride | 0.60% |
| Glycerol | 1.00% |

The oil phase A is heated to 80° C., then components B are added. The water phase C is added with stirring using a high-speed dispersing tool. The emulsion is stable at a storage temperature of +50° C. for at least 6 weeks. The emulsion is stable after 5 cycles in a swing test (−12° C. /+50° C.).

1. A cosmetic composition comprising a fragrance and an emulsifier wherein the emulsifier comprises an ester of an alkenylsuccinic anhydride of formula (2): 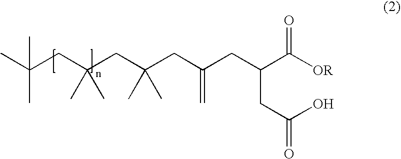
wherein n is equal to or greater than 4, and R is derived from a mono-functional alcohol selected from the group consisting of an ethoxylated monoalcohol, a propoxylated monoalcohol, a fatty alcohol ethoxylate, methyl glycol and methyltriglycol.

2. The cosmetic composition of in claim 1, which is in the form of an emulsion.

3. The cosmetic composition of in claim 1 which is in the form of a water-in-oil emulsion.

4. The cosmetic composition of claim 1, which is in the form selected from the group consisting of an ointment, a cream, a lotion, a gel, and a spray.

5. The cosmetic composition of claim 1, wherein the cosmetic composition comprises 0.1 to 8% by weight of the emulsifier.

6. A cosmetic composition comprising a fragrance and an emulsifier wherein the emulsifier comprises an ester of alkenylsuccinic anhydride of formula (2a): 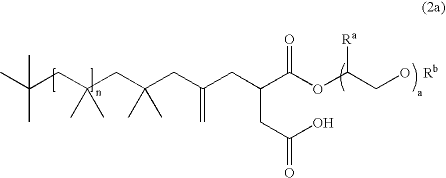
wherein n is equal or greater than 4,
R^{a} is H or methyl,
a is an integer of from 1 to 10, and
R^{b} is an alkyl group comprising 1 to 22 carbon atoms.

7. The cosmetic composition of claim 6, wherein R^{b} is an alkyl group comprising 1 to 12 carbon atoms.

8. The cosmetic composition of claim 6, wherein R^{b} is an alkyl group comprising 1 to 4 carbon atoms.

9. The cosmetic composition of claim 6, wherein R^{a} is H.

10. The cosmetic composition of claim 6, wherein a is an integer of from 1 to 7.

11. The cosmetic composition of claim 6, wherein a is 3, R^{a} is H, and R^{b} is methyl.

12. The cosmetic composition of claim 11, wherein n is 15.

13. The cosmetic composition of claim 6, wherein in a is 7, R^{a} is H and R^{b} is methyl.

14. The cosmetic composition of claim 13, wherein n is 15.

15. A method for treating human skin, said method comprising contacting skin with the cosmetic composition of claim 1.

16. A method for treating human skin, said method comprising contacting the human skin with the cosmetic composition of claim 6.
